(19) United States
(12) Patent Application Publication
(10) Pub. No.: US 2018/0185603 A1
(43) Pub. Date: Jul. 5, 2018

(54) OXYGEN BIOFEEDBACK DEVICE AND METHODS

(71) Applicants: Chris Salvino, (US); Scott White, (US)

(72) Inventors: Chris Salvino, Scottsdale, AZ (US); Scott White, Scottsdale, AZ (US)

(21) Appl. No.: 15/739,968

(22) PCT Filed: Jun. 24, 2016
(86) PCT No.: PCT/US2016/039440

§ 371 Date: Dec. 26, 2017

Related U.S. Application Data
(60) Provisional application No. 62/189,658, filed on Jul. 7, 2015.
(60) Provisional application No. 62/183,902, filed on Jun. 24, 2015.

(51) Int. Cl. A61M 16/10; A61M 16/06

(52) U.S. Cl. CPC A61M 16/1005; A61M 16/0677; A61B 5/486; A61M 2230/205; A61M 2230/432; A61M 2202/0208

(57) ABSTRACT

Supplemental oxygen is used by millions of people each year in hospitals and at home. The device and methods described allow people on supplemental oxygen through a feedback loop to optimize their blood oxygen level by measuring oxygen and/or carbon dioxide and/or other related gases in the blood. Because the device and methods optimize the level of supplemental oxygen and/or carbon dioxide and/or other related gases, complications (from too much or too little oxygen and/or carbon dioxide) including death can be prevented. In addition, users can reduce their costs by reducing the amount of oxygen needed as well as labor costs. Additionally, helicopters, ambulance, and mobile surgical sites can reduce weight in critical situations. In addition, the device and methods described also allow patients on ventilators through a feedback loop to optimize ventilation by measuring carbon dioxide in the blood; which can reduce complications, and reduce labor costs. Finally, the device and methods provides a warning system when the oxygen supply is compromised, has or is exhausted.

### CROSS-REFERENCE TO RELATED APPLICATION

This is a national phase application of PCT Application PCT/US16/39440 filed Jun. 24, 2016 which claims priority to U.S. provisional application No. 62/189,658 filed Jul. 7, 2015 and U.S. provisional application No. 62/183,902 filed Jun. 24, 2015 the contents of which are hereby incorporated by reference.

### FIELD OF THE INVENTION

The present invention is in the field of supplemental oxygen devices and pertains to biofeedback measurements which are used for regulating the rate and concentration of supplemental oxygen for those patients about to be, or who have been placed on, supplemental oxygen. In addition, the present invention is in the field of mechanical ventilators and pertains to biofeedback measurements of which are used to regulate components of minute ventilation and/or minute ventilation in general—as well as regulating the rate and concentration of supplemental oxygen. Finally, the present invention is in the field of a warning system for oxygen supply devices to warn patients and medical care providers when the oxygen supply is compromised, has or is exhausted.

### BACKGROUND

Patients with acute injury or illness who normally are not on oxygen may be started on oxygen in a ground or air ambulance, clinic, emergency room or acute care facility to improve their oxygen levels in their body to temper the side effects of low oxygen from the illness or injury. The oxygen delivered is usually in the form of a face mask, nasal cannula or ventilator circuit. The amount of oxygen, given to the patient is adjusted, if at all, intermittently and manually. For example, a patient with an acute pneumonia is placed on oxygen and many times, but not always, a pulse oximeter placed on his/her finger to measure cutaneous oxygen saturation (SpO2). Frequently a medical provider, usually a respiratory therapist, will then intermittently (sometimes just 1× a day) adjust the amount of oxygen delivered to the patient based on spot readings of the SpO2; of note, in between the spot readings the needs of the patient may vary widely resulting in too

much or too little oxygen (both of which can cause complications) being delivered most of the time the patient is on oxygen.

Approximately 67,000 ventilators exist in the United States for those patients requiring mechanical ventilation for various reasons. Ventilators have gotten more advanced with built in microprocessors; however, two key processes of patient care on ventilators remain intermittent and manual/labor intensive—portions of oxygenation and ventilation. Oxygenation—doctors normally place an order for a patient to be placed on a certain amount of delivered oxygen and a medical provider intermittently monitors their cutaneous oxygen saturation levels and/or invasive oxygen levels via a blood sample called a blood gas. Based on the oxygen levels measured the medical provider could leave the delivered oxygen amount the same, turn it higher or lower—of note, in between the spot readings the needs of the patient may vary widely most of the time resulting in too much or too little oxygen (both of which can cause complications) being delivered while the patient is on oxygen. In addition, this manual method of adjusting the oxygen delivery is labor intensive and can have human errors. Ventilation—doctors normally place an order for a patient to be placed on a certain amount of minute ventilation, which is respiratory rate/minute multiplied by the tidal volume, and a medical provider intermittently monitors their cutaneous carbon dioxide saturation levels and/or invasive carbon dioxide levels via a blood sample called a blood gas. Based on the carbon dioxide levels measured the medical provider could leave the minute volume the same, or adjust it higher or lower—of note, in between the spot readings the needs of the patient may vary widely most of the time resulting in too much or too little minute ventilation (both of which can cause complications) being delivered while patient is on a ventilator.

Supplemental oxygen can be supplied to patients either through bottled tanks or liquid oxygen. Liquid oxygen is converted to gaseous oxygen before reaching the patient. Typically, bottled tanks are used for out of hospital use and transport of patients and liquid oxygen is used primarily in hospitals. Both bottled oxygen and liquid supplies can become exhausted resulting in serious harm or death to patients if not recognized; this problem is much more likely to happen with bottled oxygen than liquid, but it still can happen with liquid oxygen supplies. In addition, oxygen supply (bottled or liquid) fittings can become dislodged resulting in serious harm or death to patients if not recognized as the patient will be without oxygen. There are no warning systems if the bottled oxygen supply is exhausted or near exhausted, and there are no warning systems for either liquid or bottled oxygen if the oxygen fittings become dislodged. There are warming systems at the liquid oxygen source (such as a supply room of a hospital) if the liquid oxygen system is being depleted, but even then there is no warning system at the patient level/near the patient.

More than 50 million people undergo surgery or invasive procedures in which oxygen is used during and/or after the procedure. Most of the time after surgery the weaning, or just removing, of oxygen off the patient is based on spot checks of Sp02 and/or just evaluating the patient clinically as he/she recovers from the anesthetic. Of note, in between the spot readings the needs of the patient may vary widely resulting in too much or too little oxygen (both of which can cause complications) being delivered the most of the time the patient is on oxygen.

In the US today there exists approximately 1 million people who suffer from chronic hypoxemia as a result of having a chronic obstructive pulmonary disease (COPD). Presently there is no cure for this condition, however the detrimental impact of chronic hypoxemia is mitigated by the prescription of long term oxygen therapy (LTOT). The continuous inhalation of low flows of oxygen, typically 2-3 lpm, from a nasal cannula increases the concentration of oxygen that the patient is breathing. It is estimated that for each 1 lpm (liter per minute) flow, the overall inhaled concentration rises by 3-4%. The increase in oxygen concentration compensates for the poor function of the patient's lungs in absorbing the oxygen.

Generally when a patient is diagnosed with chronic hypoxemia, oxygen is prescribed at a fixed flow rate based on a 20 minute titration in the doctor's office. During the test, the patient's blood oxygen saturation is measured by either using an invasive blood gas analyzer or a non-invasive device known as the pulse oximeter. While measuring the blood saturation (SpO2), the patient may be asked to walk on a treadmill so as to measure their need for supplemental oxygen while exerting themselves. Based on this brief test, a fixed flow of oxygen, is prescribed. The patient may be advised to increase the flow rate of oxygen during the exertion, for example while climbing stairs, while sleeping or if they feel short of breath. In many cases the patient is just prescribed a flow rate of 2 lpm and then asked to come back if they continue to feel the side effects of hypoxemia which can manifest themselves as shortness of breath, headaches, nausea, etc.

Chronic obstructive pulmonary disease (COPD), also known as chronic obstructive lung disease (COLD), and chronic obstructive airway disease (COAD), among others, is a type of obstructive lung disease characterized by chronically poor airflow. It typically worsens over time. The main symptoms include shortness of breath, cough, and sputum production. Most people with chronic bronchitis have COPD. Tobacco smoking is the most common cause of COPD, with a number of other factors such as air pollution and genetics playing a smaller role. In the developing world, one of the common sources of air pollution is from poorly vented cooking and heating fires. Long-term exposure to these irritants causes an inflammatory response in the lungs resulting in narrowing of the small airways and breakdown of lung tissue known as emphysema. The diagnosis is based on poor airflow as measured by lung function tests. In contrast to asthma, the airflow reduction does not improve significantly with the administration of medication. COPD can be prevented by reducing exposure to the known causes. This includes efforts to decrease rates of smoking and to improve indoor and outdoor air quality. COPD treatments include quitting smoking, vaccinations, rehabilitation, and often inhaled bronchodilators and steroids. Some people may benefit from long-term oxygen therapy or lung transplantation. In those who have periods of acute worsening, increased use of medications and hospitalization may be needed. Worldwide, COPD affects 329 million people or nearly 5% of the population. In 2013, it resulted in 2.9 million deaths up from 2.4 million deaths in 1990. The number of deaths is projected to increase due to higher smoking rates and an aging population in many countries. It resulted in an estimated economic cost of $2.1 trillion in 2010.

Chronic hypoxemic patients may be prescribed oxygen to breathe 24 hours per day or may only require oxygen while ambulating. If a patient needs to breathe oxygen even while resting, they will be given a stationary oxygen generating unit in their home, or rarely bottled oxygen, which can be set to produce 0 to 5 lpm of supplemental oxygen. Generally, the units today are manually set by the patient to the prescribed flowrate. If a patient requires oxygen while ambulating, they will typically carry small high pressure oxygen cylinders or small refillable liquid oxygen dewars. Recently, small portable oxygen generators have also been introduced into the market but they suffer from drawbacks of being significantly heavier and short battery life. These devices also would be manually set by the patient to deliver oxygen at the prescribed flow rate. Due to the expense of providing oxygen in small cylinders as well as dewars for ambulation, the need to conserve the oxygen flow and efficiently utilize what was available was addressed by the development of oxygen conserving devices. These devices only deliver short pulses of oxygen at the beginning of the patient's inhalation. By not delivering oxygen during exhalation or the later period of inhalation, the oxygen which would have had no impact on increasing the patient's oxygen saturation is conserved. There now exists both pneumatic and electronic oxygen conserving devices which can achieve oxygen conserving ratios from 2:1 to 6:1 compared to the delivery of continuous oxygen flow. The higher conservation ratios can only be achieved by the electronic devices since they can be programmed to skip breaths so that oxygen pulse is only delivered every other breath. Electronic devices cannot be used on all ambulating patients since their high conservation ratios can actually result in poor oxygen saturation for the patient particularly during periods of high ambulation. Of note, since there is no biofeedback system in place, no matter how oxygen is delivered for those with chronic hypoxemia, the needs of the patient may vary widely throughout the day, but the amount of oxygen being delivered remains fixed, resulting in too much or too little oxygen (both of which can cause complications) being delivered most of the time the patient is on oxygen. In addition, if the patient is using bottled oxygen, they will end up exhausting the bottle supply of oxygen sooner due to lack of accuracy of delivery of oxygen as there is no biofeedback system in place.

U.S. Pat. No. 4,889,116 by Taube in 1986 describes an adaptive controller, which utilizes a pulse oximeter to measure blood oxygen saturation (SpO2). This measurement would be used to calculate the necessary Fl02 to maintain a preset saturation level.

U.S. Pat. No. 5,365,922 by Raemer describes a closed loop non-invasive oxygen saturation control system which uses an adaptive controller for delivering a fractional amount of oxygen to a patient. Features of the control algorithm include a method for recognizing when pulse oximeter values deviate significantly from what should be expected. At this point the controller causes a gradual increase in the fractional amount of oxygen delivered to the patient. The feedback control means is also disconnected periodically and the response of the patient to random changes in the amount of oxygen delivered is used to tune the controller response parameters.

U.S. Pat. No. 5,682,877 describes a system and method for automatically selecting an appropriate oxygen dose to maintain a desired blood oxygen saturation level is disclosed. The system and method are particularly suited for use with ambulatory patients having chronic obstructive lung disease or other patients requiring oxygenation or ventilation. In one embodiment, the method includes delivering a first oxygen dose to the patient while repeatedly sequencing through available sequential oxygen doses at predetermined time intervals until the current blood oxygen saturation level of the patent attains the desired blood oxygen saturation levels. The method then continues with delivering the selected oxygen dose to the patient so as to maintain the desired blood oxygen saturation level.

U.S. Pat. No. 6,192,883 B1 describes an oxygen control system for supplying a predetermined rate of flow from an oxygen source to a person in need of supplemental oxygen comprising in input manifold, an output manifold and a plurality of gas conduits interconnecting the input manifold to the output manifold. The oxygen source is arranged in flow communication with the input manifold, and a needle valve is positioned in flow control relation to each of the conduits so as to control the flow of oxygen from the input manifold to the output manifold. A plurality of solenoid valves, each having a first fully closed state corresponding to a preselected level of physical activity of the person and a second, fully open state corresponding to another preselected level of physical activity of the person, are positioned in flow control relation to all but one of the conduits. Sensors for monitoring the level of physical activity of the person are provided, along with a control system that is responsive to the monitored level of physical activity, for switching the solenoids between the first state and the second state. A method for supplying supplemental oxygen to a person according to the level of physical activity undertaken by that person is also provided.

The use of feedback loops is well known in many industries. A proportional-integral-derivative controller (PID controller) is a control loop feedback mechanism (controller) widely used in industrial control systems. A PID controller calculates an error value as the difference between a measured process variable and a desired setpoint. The controller attempts to minimize the error by adjusting the process through use of a manipulated variable. The PID controller algorithm involves three separate constant parameters, and is accordingly sometimes called three-term control: the proportional, the integral and derivative values, denoted P, I, and D. Simply put, these values can be interpreted in terms of time: P depends on the present error, I on the accumulation of past errors, and D is a prediction of future errors, based on current rate of change. The weighted sum of these three actions is used to adjust the process via a control element such as the position of a control valve, a damper, or the power supplied to a heating element. Alternative micro-controllers are known in the industry such as a field-programmable gate array (FPGA) is an integrated circuit designed to be configured by a customer or a designer after manufacturing—hence “field-programmable.” The FPGA configuration is generally specified using a hardware description language (HDL), similar to that used for an application-specific integrated circuit (ASIC). (Circuit diagrams were previously used to specify the configuration, as they were for ASICs, but this is increasingly rare.) A Spartan FPGA from Xilinx. FPGAs contain an array of programmable logic blocks, and a hierarchy of reconfigurable interconnects that allow the blocks to be “wired together,” like many logic gates that can be inter-wired in different configurations. Logic blocks can be configured to perform complex combinational functions, or merely simple logic gates like AND and XOR. In most FPGAs, logic blocks also include memory elements, which may be simple flip-flops or more complete blocks of memory.

These and all other referenced patents are incorporated herein by reference in their entirety. Furthermore, where a definition or use of a term in a reference, which is an incorporated reference here, is inconsistent or contrary to the definition of that term provided herein applies and the definition of that term in the reference does not apply.

### SUMMARY OF THE INVENTION

In view of the shortcomings of the prior art, it is the object of this invention to provide a method to reduce supplemental oxygen use and to make the use of supplemental oxygen more accurate by measuring oxygen and/or ventilation adjustment parameters (“OVAP”) via different sensors for blood gas and tissue gas concentrations.

Another object of the invention is to provide a method to monitor patients post operatively and post conscious sedation and to wean the patient off of supplemental oxygen in a quick and safe manner by monitoring one or more OVAP and through a feedback loop to adjust the amount and flow of oxygen delivered based on the OVAP measured.

Another object of the invention is to reduce the total amount of weight of a mobile oxygen delivery system (liquid or bottled-“gaseous” oxygen) by monitoring various OVAP and through the feedback loop the accuracy of the oxygen delivery to the patient will be improved and consequently reduce the amount of oxygen used; ultimately reducing weight of oxygen needed.

Another object of the invention is to provide a device for measuring OVAP that provides a feedback loop to automatically increase or decrease the supply of supplemental oxygen.

Another object of the invention is to provide a device for measuring OVAP that can be used to determine if an athlete has used illegal doping strategies.

Another object of the invention is to provide a device for measuring OVAP for continuous monitoring.

Another object of the invention is to provide a device for measuring OVAP for monitoring disease states such as diabetes or obstructive sleep apnea.

Another object of the invention is to provide a device for measuring OVAP for continuous monitoring that communicates with a mobile device for recording, tracking, and sharing of OVAP data as well as oxygen delivery needs.

Another object of the invention is to provide a device for measuring OVAP for patients on a ventilator and providing a feedback loop to continuously adjust the amount of minute ventilation.

Another object of the invention is to provide a device for measuring OVAP for patents on a ventilator that provides a feedback loop to automatically increase or decrease the supply of supplemental oxygen.

Another object of the invention is to provide a device for measuring OVAP for continuous monitoring of acutely ill or injured patients who have been placed on supplemental oxygen with a goal of providing a feedback loop to continuously adjust the amount of oxygen delivered through a face mask, nasal cannula or similar device.

Another object of the invention is to provide a device for measuring when the oxygen supply is depleting and/or depleted and/or dislodged/disconnected and to provide an audible and/or visible warning to the medical provider and/or patient.

Another object of the invention is to provide a device that is compact, ruggedized and has components such as a microprocessor that are robust and can be sealed from the external environment to be water resistant and sand resistant.

Further objects and advantages of the invention will become apparent to those skilled in the art upon reading and consideration of the following description of a preferred embodiment and the accompanying drawings.

### BRIEF DESCRIPTION OF THE DRAWINGS

FIG. 1 shows a preferred embodiment of a person connected to a sensor on the skin of their hand or other body part, the sensor measuring STO2 and/or SpO2 and/or PCO2 (all 3 are a subset of the entire list of OVAP and are here as examples of OVAP that can be measured), the sensor connected to a controller, the controller connected to an oxygen supply and capable of adjusting the dose of supplemental oxygen to a person.

FIG. 2 shows a preferred embodiment of a person connected to a sensor in an artery or vein, the sensor measuring STO2 and/or SpO2 and/or PCO2 (all 3 are a subset of the entire list of OVAP and are here as examples of OVAP that can be measured), the sensor connected to a controller, the controller connected to an oxygen supply and capable of adjusting the dose of supplemental oxygen to a person.

### DETAILED DESCRIPTION

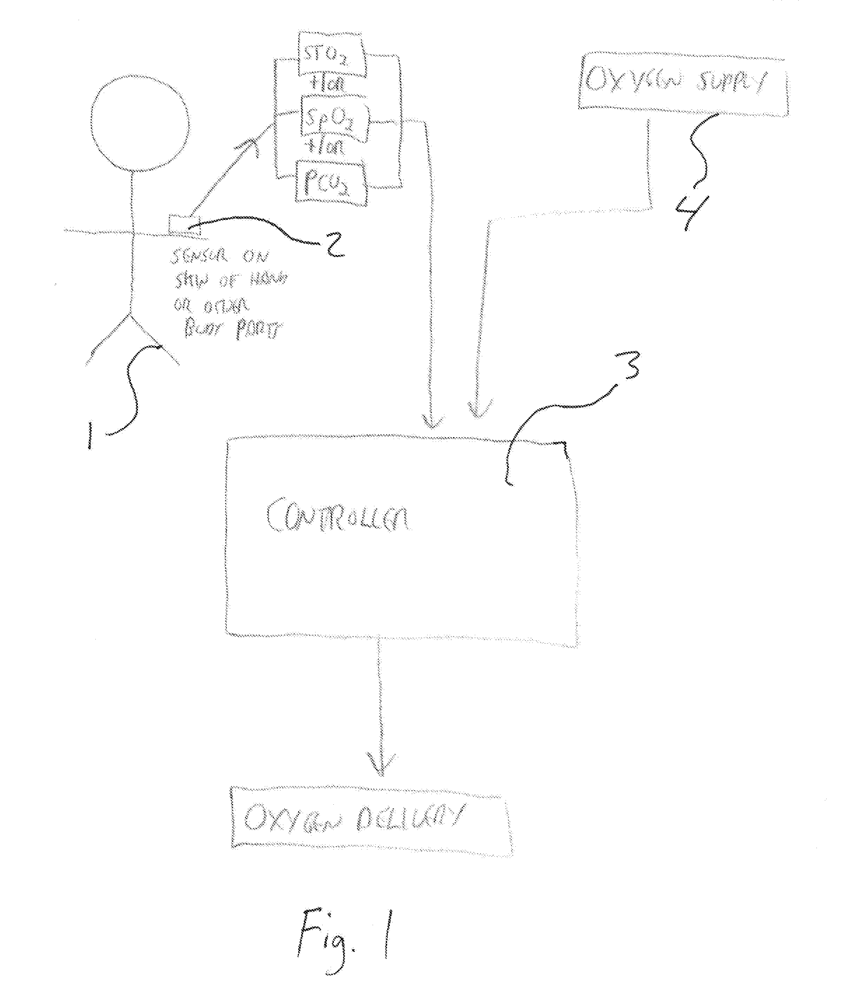

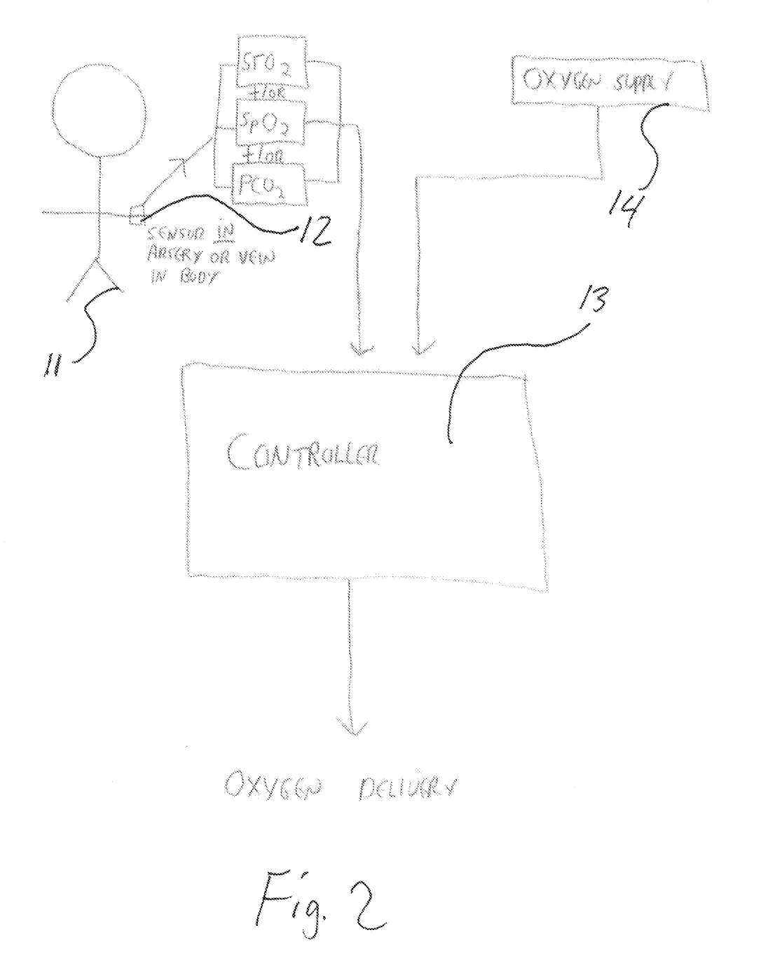

Referring now to the drawings wherein the showings are for purposes of illustrating a preferred embodiment of the present invention and not for purposes of limiting the same. The present invention is primarily focused on non-invasive cutaneous gas sensors and methods; however, it should be understood that the sensors and methods disclosed herein could be adapted to measure and monitor blood too. Throughout the detailed description the invention discloses sensors and methods of sensing gases in tissue and blood, the most common measurement being oxygen saturation via an oximeter and often described as SpO2. There are numerous sensors that are capable of measuring other tissue and blood gas concentrations. It should be understood throughout that the present invention may utilize one or more various sensors for measuring oxygen and/or ventilation adjustment parameters (“OVAP”) in each embodiment and that specific examples are given for clarity and not to limit the scope of the invention unless otherwise expressly stated. It should be understood throughout that the present invention may utilize one or more various sensors for measuring oxygen and/or ventilation adjustment parameters (“OVAP”). OVAP include at least oxygen saturation, carbon dioxide, partial pressure of oxygen in blood, and other parameters for the fetus, child and/or adult and measured either across the skin (cutaneous), or via invasive blood sampling of venous or arterial blood or via invasive blood measuring of venous or arterial blood. FIG. 1 shows a user 1 with a sensor 2 on the user's skin, the sensor 2 further connected to a controller 3. The controller 3 has an oxygen supply 4 to be delivered to the user via face mask, nasal cannula, ventilator or similar device. FIG. 2 shows a user 11 with a sensor 12 in the user's artery or vein, the sensor 12 further connected to a controller 13. The controller 13 has an oxygen supply 14 to be delivered to the user via face mask, nasal cannula, ventilator or similar device.

1. Feedback Sensor and Method

The present invention contemplates the use of one or more OVAP sensors to increase accuracy and detect disease states based on known blood and tissue gas parameters that fall outside the normal range. For example, StO2 can be monitored. Tissue oxygenation monitor measures tissue optical attenuation values at 680, 720, 760, and 800 nm. The light in the InSpectra StO2 Cable contains the four wavelengths of light used for the InSpectra™ StO2 System Measurement. The maximum depth of the tissue volume sampled is estimated to equal the distance between the sensor's send and receive fibers. Cui, Kumar, and Chance (1991) confirmed that the mean measurement depth into the tissue is half of the sensor spacing. The InSpectra™ StO2 Sensor 1615: 15 mm is designed to measure the proper depth of the tissue sampled in the thenar eminence. There are two light points on the face of the sensor that send and receive a signal from the patient's tissue. The comparison of the receive signal from the patient and the receive feedback signal within the oximitor is processed into a second derivative attenuation spectrum using a fixed wavelength gap point difference calculation. The resultant second derivative attenuation spectrum is sensitive to deoxyhemoglobin and oxyhemoglobin absorption. The absorption spectrum of light returned from a tissue sample varies mainly with oxyhemoglobin and deoxyhemoglobin concentration; other chromophores have less effect.

Other advanced technology is that developed by Modulate Imaging, Inc. of Irvine, Calif. They have developed non-invasive Spatial Frequency Domain Imaging technology to determine gas levels non-invasively. Harvey, S L et. al. discloses a new platinum/platinum ring-disc microelectrode for monitoring tissue perfusion is a mass transport mechanism that describes the movement of respiratory gases, nutrients and metabolites in tissue. The sensor's capability of detecting perfusion at the cellular level in a continuous fashion is unique. This sensor will provide insight into the way nutrients and metabolites are transported in tissue especially in cases were perfusion is low such as in wounds or ischemic tissue, Conf Proc IEEE Eng Med Biol Soc. 2007; 2007:2689-92. Additional sensor and techniques have been described by Nguyen J T, et al. in A novel pilot study using spatial frequency domain imaging to assess oxygenation of perforator flaps during reconstructive breast surgery, Ann Plast Surg. 2013 September; 71 (3):308-15. The results were Spatial frequency domain imaging was able to measure tissue oxyhemoglobin concentration (ctO2Hb), tissue deoxyhemoglobin concentration, and tissue oxygen saturation (stO2). Images were created for each metric to monitor flap status and the results quantified throughout the various time points of the procedure. For 2 of 3 patients, the chosen flap had a higher ctO2Hb and stO2. For 1 patient, the chosen flap had lower ctO2Hb and stO2. There were no perfusion deficits observed based on SFDI and clinical follow-up.

In a preferred embodiment, device measures SpO2 and adjusts supplemental oxygen supply upward or downward depending on a physician setting at least one set point for each OVAP used. After a physician sets the device setpoints a PID controller 3 will adjust oxygen delivery based on SpO2 to the setpoint. For example, for a patient with COPD, the physician may target the setpoint of SpO2 to 92-95% but no higher because a high SpO2 could cause injury to the lung alveoli. For example, normal PaCO2 typically ranges from 35 to 45 mm HG; a patient who has had surgery and is recovering could end up with elevated PaCO2 and decreased SpO2 if too much narcotics had been given; the controller would increase the amount of oxygen to return the SpO2 to levels set by the physician and an alarm would go off if the PaCO2 went outside of the set parameters. Another example is a COPD patient in which giving too much oxygen could lead to the CO2 climbing above 45 and then the feedback loop would decrease the oxygen until the CO2 returned to normal.

In an alternative preferred embodiment, device measures SpO2 and PaCO2 and adjusts supplemental oxygen supply upward or downward depending on a physician setting at least one set point for each OVAP used. After a physician sets the device setpoints a PID controller 3 will adjust oxygen delivery based on SpO2 to the setpoint. For example, for a patient with COPD, the physician may target the setpoint of SpO2 to 92-95% but no higher because a high SpO2 could cause injury to the lung alveoli. For example, normal PaCO2 typically ranges from 35 to 45 mm HG; a patient who has had surgery and is recovering could end up with elevated PaCO2 and decreased SpO2 if too much narcotics had been given; the controller would increase the amount of oxygen to return the SpO2 to levels set by the physician and an alarm would go off if the PaCO2 went outside of the set parameters. Another example is a COPD patient in which giving too much oxygen could lead to the CO2 climbing above 45 and then the feedback loop would decrease the oxygen until the CO2 returned to normal.

In an alternative preferred embodiment, device measures SpO2 and/or PaCO2 and/or other OVAP values and adjusts supplemental oxygen supply upward or downward depending on a physician setting at least one set point for each OVAP used. After a physician sets the device setpoints a PID controller 3 will adjust oxygen delivery based on SpO2 to the setpoint. For example, for a patient with COPD, the physician may target the setpoint of SpO2 to 92-95% but no higher because a high SpO2 could cause injury to the lung alveoli. For example, normal PaCO2 typically ranges from 35 to 45 mm HG; a patient who has had surgery and is recovering could end up with elevated PaCO2 and decreased SpO2 if too much narcotics had been given; the controller would increase the amount of oxygen to return the SpO2 to levels set by the physician and an alarm would go off if the PaCO2 went outside of the set parameters. Another example is a COPD patient in which giving too much oxygen could lead to the CO2 climbing above 45 and then the feedback loop would decrease the oxygen until the CO2 returned to normal.

2. Weaning

In a preferred embodiment of the present invention the device would monitor a patient's SpO2 and/or PaCO2 or other OVAP sensor after an anesthetic procedure or other procedure that requires supplemental oxygen. The PID controller of the device is set by the physician to a setpoint of SpO2, for example, of between 85-95% saturation. The PID controller regulates the rate of oxygen flow from a source. The physician can, for example, make the settings different for patients with different chronic diseases to target weaning a patient form supplemental oxygen from ten to thirty minutes, although up to one hour would be acceptable for patients that have had general anesthesia. The target time will be set by a health care provider such as a physician. When the PID controller detects the rate of oxygen decrease the device can make corrections so that the patient does not under go long periods of SpO2 below 85-92%. This allows hospitals to be more efficient because nurses and doctors do not have to spot check the patient while weaning after a procedure. Additionally, the patient is safe because the device has an alarm if the SpO2 and PaCO2 are out of the specified range for too long or too far out of range that the PID controller predicts the patient needs intervention greater than the maximum supply of oxygen from the oxygen source.

3. Mobile

In a preferred embodiment of the present invention the device is compact and ruggedized for mobile applications. For example, helicopters and ambulance have limited space and limited load capacity. The present invention uses a small PID microprocessor that is robust and can be sealed from the external environment to be water resistant and sand resistant. Because the device is small and does not weigh much, the oxygen that is saved through efficiency can reduce the size of oxygen bottles utilized in mobile applications. Additionally, the device can reduce the costs associated with having to refill oxygen bottles frequently.

4. Sleep Apnea and Health Related Monitoring App

In a preferred embodiment of the present invention the device can be wrist worn or attachable to clothing, i.e. wearable for continuous blood monitoring. Additionally, the device could incorporate Photoplethysmogram sensors to measure pulse rate. The device would additionally have a BlueTooth® or other WiFi communication means that would link with a smart device such as an Android® or iPhone® to monitor and record OVAP levels as well as oxygen usage. This would be very useful for remote monitoring of patients suspected of having obstructive sleep apnea. A software app loaded on the smart device would store the data and create visual data charts for easily understandable conditions. The software app loaded on the smart device may also directly transmit OVAP levels to a physician, hospital or other identified health care provider. For example the American Academy of Sleep Medicine (AASM) was assembled to produce a clinical guideline from a review of existing practice parameters and available literature. Journal of Clinical Sleep Medicine, Vol. 5, No. 3, 2009. The app would incorporate the clinically relevant apnea events and a microphone on the smart device could be used to detect snoring.

5. IOC Anti Doping Monitor

In a preferred embodiment of the present invention the device could be used by agencies like USADA, the United States Anti-Doping Agency to create a standard of normal recovery times for oxygen saturation recovery. For example, an athlete could be placed in a hypo baric (or reduced oxygen atmosphere) chamber for ten minutes to thirty minutes and determine if the athletes response is outside the normal range of responses in order to detect artificial treatments. Also, the chamber could be introduced with normal air or hyper oxygenated or under hyperbaric conditions and the response to OVAP responses would be indicative of artificial treatments. For example, the hypo baric chamber could have a preconditioning setting between ten to thirty minutes. Then the introduction of normal air, hyperbaric air, or oxygen enriched air would be introduced into the chamber. If the athletes OVAP fell outside of the normal recovery of oxygen saturation or other OVAP metrics it would signal an artificial treatment.

6. Ventilators

Health care providers often use elevated arterial pCO2 (“PaCO2”) as an indicator of incipient respiratory failure. In this regard, the determination of PaCO2 is useful in optimizing the settings on ventilators and detecting life-threatening OVAP changes in an anesthetized patient undergoing surgery. In a preferred embodiment of the present invention the device is configured for continuous monitoring of SpO2 and/or PaCO2 or other OVAP sensor. Oxygen delivered to the patient directly through the ventilator circuit would be continuously adjusted to optimize the set point or range of SpO2 and/or PaCO2 or other OVAP sensor set by the medical provider. Minute ventilation, via its subsets such as tidal volume and/or respiratory rate per minute, would be continuously adjusted to optimize the set point or range of SpO2 and/or PaCO2 or other OVAP sensor set by the medical provider. Thus, the invention can continuously regulate supplemental oxygen delivery as well as minute ventilation by constant biofeedback from OVAP. Acute maintenance.

7. Continuous Monitoring

In a preferred embodiment of the present invention the device could continuously monitor a patient admitted to an air or ground ambulance, clinic, emergency room or acute care facility and the amount of oxygen delivered to the patient directly through the face mask, nasal cannula or similar device wound be continuously adjusted to optimize the set point or range of SpO2 and/or PaCO2 or other OVAP sensor set by the medical provider. In this case the patient may or may not be on supplemental oxygen but the data from the continuous monitoring could be stored on flash memory in the device and available for real-time transmission to a facility server for alarm monitoring. Alternatively, the stored data could be download and charted just prior to a patient's examination with a physician, nurse or other health care provider.

8. Fail-Safe Mechanism

In all of the above disclosed embodiments the controller would have a fail-safe mechanism for detecting failure (either exhaustion of the oxygen supply or a disconnection of the fittings) of supplemental oxygen delivery. The controller default position in the fail-safe mode would be to open up oxygen from the source and alarm. The alarm would be audible and/or visual. The alarm would be at the site of the device use as well as remote alarm via communication technology such as WiFi and BlueTooth®. An additional safety feature is the ability to test oxygen delivery in line from the oxygen source on route to the patient and alarm if the oxygen source were depleted or close to being depleted, for example if there was a pressure drop in a pressure regulator at the oxygen source that would trigger an alert that the oxygen supply was getting low.

9. Energy Supply, Recording and Data Sharing

It should be understood from the context that the above embodiments could be powered by hardwire, disposable battery, rechargeable battery, USB compatible for rechargeable battery. Additionally, the embodiments could incorporate various memory for recording data and either sharing in real time or saved on an SD memory card for later transmission.

Additional modifications and improvements of the present invention may also be apparent to those skilled in the art. Thus, the particular combination of parts described and illustrated herein in intended to represent only one embodiment of the invention, and is not intended to serve as limitations of alternative devices within the spirit and scope of the invention.

What is claimed:

1. An apparatus with dual sensing means for automatically controlling and conserving the delivery of oxygen to a patient comprising: an oxygen supply; means having a first non-invasive sensor to measure blood hemoglobin saturation (SpO2) of a patient and a second non-invasive sensor to measure PaCO2 of the patient; means for providing desired range of OVAP saturation for the patient; first control means adapted for identifying a first error signal representing the difference between at least one setpoint level of the range and a signal representing the measurement of the hemoglobin saturation (SpO2)); second control means adapted for identifying a first error signal representing the difference between at least one setpoint level and a signal representing the measurement of the PaCO2; means for responding to the hemoglobin saturation (SpO2) and PaCO2 setpoints to increase or decrease oxygen flow rate.

2. The apparatus of claim 1 wherein the oximeter means comprises a pulse oximeter adapted to be worn on a patients wrist, other body parts, or clothes and having a first probe from the oximeter contacting the patients skin and measure hemoglobin saturation (SpO2) and a second probe form the sensor adapted to adhere to the skin of the thenar eminence, or other appropriate body parts, to measure PaCO2.

3. The apparatus of claim 1 further comprising an alarm means adapted to indicate any default in the operation of the apparatus.

4. The apparatus of claim 1 further compromising au audio and/or visual alarm adapted to indicate loss of oxygen pressure from the oxygen source indicating either a fitting disconnection, exhaustion of the oxygen supply, near exhaustion of the oxygen supply, or inadvertent turning off of the oxygen supply.

5. A method for delivering and controlling oxygen to a patient from an oxygen supply while effectively conserving said oxygen supply, comprising the steps of: a) providing a supply of oxygen from an oxygen source; b) providing a desired range with at least one set point signal for the blood oxygen hemoglobin saturation (SpO2) of a patient; c) measuring the blood oxygen hemoglobin saturation (SpO2) in the patient and providing said measured value as an SpO2 signal; d) generating a first error signal by subtracting the setpoint signal from the measured blood hemoglobin saturation signal; e) providing a desired range with at least one set point signal for the PaCO2 of a patient; f) measuring the PaCO2 in the patient and providing said measured value as a PaCO2 signal; g) generating a second error signal by subtracting the setpoint signal from the measured blood hemoglobin saturation signal; h) generating an oxygen flow setpoint signal by combining the first error signal and the second signal; i) measuring the oxygen flow from the oxygen source and providing an oxygen flow signal; j) generating a third error signal by subtracting the oxygen flow setpoint signal from the oxygen flow signal; and k) adjusting a deliverable amount of oxygen to the patient in response to the second error signal of step.

6. The method of claim 5 wherein sensors are used to measure both the blood hemoglobin saturation (SpO2) and the PaCO2.

7. The method of claim 5 wherein the SpO2 signal and PaCO2 are provided by feed controllers wherein at least one of the controllers comprise analog or digital electrical components providing electrical input and output current signals; mechanical components providing pneumatic input and output signals; computers providing analog to digital and digital to analog converters with analog input and output lines; and artificial intelligence providing input and output signals.

8. The method of claim 5 further comprising the step of; k) indicating any default in any of the signals.

9. The apparatus of claim 1 wherein the oxygen conserver controller is a microcontroller with flash memory.

10. The apparatus of claim 1 wherein the means for detecting and using the control signal is a drive circuit coupled to a solenoid.

11. A method for weaning supplemental oxygen to a patient that effectively conserves said oxygen supply, comprising the steps of: a) providing a supply of oxygen; b) continuously measuring hemoglobin saturation (SpO2) and/or PaCO2; c) calculating a rate of supply oxygen to reduce blood hemoglobin saturation (SpO2) to 85-95 percent setpoint, or similar; and d) increase the oxygen supply rate if the blood hemoglobin saturation (SpO2) falls below the setpoint.

12. The method of claim 11 wherein the step c) the rate is calculated to achieve a hemoglobin saturation (SpO2) of 92-95 percent in thirty minutes or less. Could do 2-3% increments. Time could be 60-30 minutes; or longer.

13. The method of claim 11 wherein in step d) solenoids are used for adjusting the deliverable amount of oxygen to the patient.

14. A ventilator comprising at least one sensor for sensing OVAP and a controller with setpoints, said controller further connected to an oxygen supply controller that can increase or decrease oxygen supply and/or a controller that an increase or decrease minute ventilation.

15. The device of claim 14 wherein the controller is further connected to a pressure sensor on the oxygen supply and when a pressure in the oxygen supply drops below a predetermined value the controller will alarm.

16. An apparatus with single sensing means for automatically controlling and conserving the delivery of oxygen to a patient comprising: an oxygen supply; means having a first non-invasive sensor to measure blood hemoglobin saturation (SpO2) of a patient; means for providing desired range of OVAP saturation for the patient; first control means adapted for identifying a first error signal representing the difference between at least one setpoint level of the range and a signal representing the measurement of the hemoglobin saturation (SpO2)); means for responding to the hemoglobin saturation (SpO2) setpoints to increase or decrease oxygen flow rate.

17. An apparatus with single or multiple sensing means for automatically controlling and conserving the delivery of oxygen to a patient comprising: an oxygen supply; means having a first non-invasive sensor to measure OVAP #1 of a patient and possible second or more non-invasive sensors to measure additional OVAP #2 (where OVAP #2 represents at least 1 if not more values of OVAP beyond #1) of the patient; means for providing desired range of OVAP #1 levels for the patient; first control means adapted for identifying a first error signal representing the difference between at least one setpoint level of the range and a signal representing the measurement of the one of the OVAP #1 values; possible second or more control means adapted for identifying a first error signal representing the difference between at least one setpoint level and a signal representing the measurement of the OVAP #2 value's; means for responding to the OVAP #1 and/or OVAP #2 setpoints to increase or decrease oxygen flow rate.
